(19) United States
(12) Patent Application Publication
(10) Pub. No.: US 2013/0131625 A1
(43) Pub. Date: May 23, 2013

(54) Disposable Absorbent Article With Split Fastener

(71) Applicants: Daniel Robert Schlinz, Greenville, WI (US); Georgia Lynn Zehner, Larsen, WI (US); Eric Donald Johnson, Larsen, WI (US); Nancy Ellen Dawson, Appleton, WI (US)

(72) Inventors: Daniel Robert Schlinz, Greenville, WI (US); Georgia Lynn Zehner, Larsen, WI (US); Eric Donald Johnson, Larsen, WI (US); Nancy Ellen Dawson, Appleton, WI (US)

(21) Appl. No.: 13/299,461

(22) Filed: Nov. 18, 2011

(51) Int. Cl. A61F 13/62; A61F 13/56

(52) U.S. Cl. 604/391; 604/386

(57) ABSTRACT

An absorbent article includes front and back side panels, a closure component coupled to or integral with the front side panel, an anchor component coupled to or integral with one of the front and back side panels, a closure landing zone coupled to or integral with the back side panel, and an anchor landing zone coupled to or integral with the side panel opposite the anchor component. The closure component is fastened to the closure landing zone to form a closure connection and the anchor component is fastened to the anchor landing zone to form an anchor connection when the article is in a wear configuration, wherein the refastenable seam includes the closure and anchor connections, and wherein the anchor connection has higher peel and shear strengths per unit area than the peel and shear strengths per unit area of the closure connection.

### BACKGROUND

The present disclosure relates generally to absorbent articles intended for personal wear, and more particularly to disposable absorbent articles.

Many absorbent articles intended for personal wear, e.g., such as diapers, training pants, feminine hygiene products, adult incontinence products, bandages, medical garments and the like are designed to be sufficiently absorbent to pull moisture from liquid body exudates including urine, menses, blood, etc. away from the wearer to reduce skin irritation caused by prolonged wetness exposure. Diapers, as an example, are typically placed and secured on a wearer using a set of primary fastening tabs, such as adhesive tabs or mechanical (e.g., hook or loop) fastening system tabs and left in place to absorb insults as well as to contain fecal waste.

Training pants, unlike diapers, typically come pre-assembled in a wear configuration to more closely resemble conventional underpants. In particular, front and back waist regions of such training pants are typically attached at a seam either permanently or refastenably (such as by a primary fastening system) to define a wear configuration of the pants having a waist opening and leg openings.

For such articles where the attachment is refastenable, such as diapers and training pants, the hook material used in the refastenable seam can be irritating to the wearer's skin, particularly on the leg opening end of the seam. Hook material of sufficient strength must be used, however, because the attachment is designed to withstand stresses placed on the attachment by movement of the wearer without pop-opens (separation of the fasteners) occurring.

### SUMMARY

There is a need, therefore, for a fastening system provided on an absorbent article such as training pants for improved resistance to pop-opens, but that is more skin friendly.

Disclosed herein is a pant design that increases the skin friendliness of a mechanical fastener side seam by providing a fastener with varying properties in the longitudinal direction, and that is provided without locking up or compromising stretch in the absorbent article.

In one aspect, an absorbent article for personal wear about a wearer's waist has a transverse axis and a longitudinal centerline. The article includes a liquid permeable inner surface for facing the wearer; an outer surface for facing away from the wearer; an absorbent body disposed therebetween; and a front waist region, a back waist region, and a crotch region extending longitudinally between and interconnecting the front and back waist regions. The article also includes a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening. The article also includes a closure component coupled to or integral with the front side panel and disposed adjacent the leg opening, an anchor component coupled to or integral with one of the front and back side panels and disposed adjacent the waist opening, a closure landing zone coupled to or integral with the back side panel, and an anchor landing zone coupled to or integral with the side panel opposite the anchor component, wherein the closure component is fastened to the closure landing zone to form an closure connection and the anchor component is fastened to the anchor landing zone to form an anchor connection when the article is in a wear configuration, wherein the refastenable seam includes the closure and anchor connections, and wherein the anchor connection has higher peel and shear strengths per unit area than the peel and shear strengths per unit area of the closure connection.

In another aspect, an absorbent article for personal wear about a wearer's waist has a transverse axis and a longitudinal centerline. The article includes a liquid permeable inner surface for facing the wearer; an outer surface for facing away from the wearer; an absorbent body disposed therebetween; and a front waist region, a back waist region, and a crotch region extending longitudinally between and interconnecting the front and back waist regions. The article also includes a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening, and wherein each of the front and back side panels has an inner surface and an outer surface. The article also includes an anchor component coupled to or integral with the inner surface of the back side panel, a closure component coupled to or integral with the outer surface of the front side panel, a closure landing zone coupled to or integral with the back side panel opposite the closure component, and an anchor landing zone coupled to or integral with the front side panel opposite the anchor component, wherein the closure component is fastened to the closure landing zone to form a closure connection and the anchor component is fastened to the anchor landing zone to form an anchor connection when the article is in a wear configuration, and wherein the anchor connection has higher peel and shear strengths per unit area than the peel and shear strengths per unit area of the closure connection.

In another aspect, an absorbent article for personal wear about a wearer's waist has a transverse axis and a longitudinal centerline. The article includes a liquid permeable inner surface for facing the wearer; an outer surface for facing away from the wearer; an absorbent body disposed therebetween; and a front waist region, a back waist region, and a crotch region extending longitudinally between and interconnecting the front and back waist regions. The article also includes a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening. The article also includes a closure component coupled to or integral with the front side panel and disposed adjacent the leg opening, the closure component including closure hook material, an anchor component coupled to or integral with one of the front and back side panels and disposed adjacent the waist opening, the anchor component including anchor hook material, a closure landing zone coupled to or integral with the back side panel, and an anchor landing zone coupled to or integral with the side panel opposite the anchor component, wherein the closure hook material is lighter and more skin friendly than the anchor hook material.

Other features of the disclosure will be in part apparent and in part pointed out hereinafter. Other objects and advantages of the present disclosure will become more apparent to those skilled in the art in view of the following description and the accompanying drawings.

### BRIEF DESCRIPTION OF THE DRAWINGS

The present disclosure will be more fully understood, and further features will become apparent, when reference is made to the following detailed description and the accompanying drawings. The drawings are merely representative and are not intended to limit the scope of the claims.

FIG. 1 is a bottom plan view of one aspect of a personal wear article in the form of a pair of training pants having an article fastening system as described herein, in an unfastened, unfolded and laid flat condition, and showing the surface of the training pants that faces away from the wearer;

FIG. 2 is a top plan view of the product of FIG. 1 showing the surface of the training pants that faces the wearer when worn and with portions cut away to show underlying features;

FIG. 3 is a partial schematic view of the article fastening system illustrated in FIGS. 1-2;

FIG. 4 is a bottom plan view of another aspect of a personal wear article in the form of a pair of training pants having an article fastening system as described herein, in an unfastened, unfolded and laid flat condition, and showing the surface of the training pants that faces away from the wearer; and

FIG. 5 is a top plan view of the product of FIG. 4 showing the surface of the training pants that faces the wearer when worn and with portions cut away to show underlying features.

Repeat use of reference characters in the present specification and drawings is intended to represent the same or analogous features or elements of the present disclosure. The drawings are representational and are not necessarily drawn to scale. Certain proportions thereof might be exaggerated, while others might be minimized.

### DETAILED DESCRIPTION

It is to be understood by one of ordinary skill in the art that the present discussion is a description of exemplary aspects of the present disclosure only, and is not intended as limiting the broader aspects of the present disclosure.

Within the context of this specification, each term or phrase below will include the following meaning or meanings.

“Bonded” refers to the joining, adhering, connecting, attaching, or the like, of two elements. Two elements will be considered to be bonded together when they are bonded directly to one another or indirectly to one another, such as when each is directly bonded to intermediate elements.

“Connected” refers to the joining, adhering, bonding, attaching, or the like, of two elements. Two elements will be considered to be connected together when they are connected directly to one another or indirectly to one another, such as when each is directly connected to intermediate elements.

“Cross direction” refers to the width of a fabric in a direction generally perpendicular to the direction in which it is produced, as opposed to “machine direction” that refers to the length of a fabric in the direction in which it is produced.

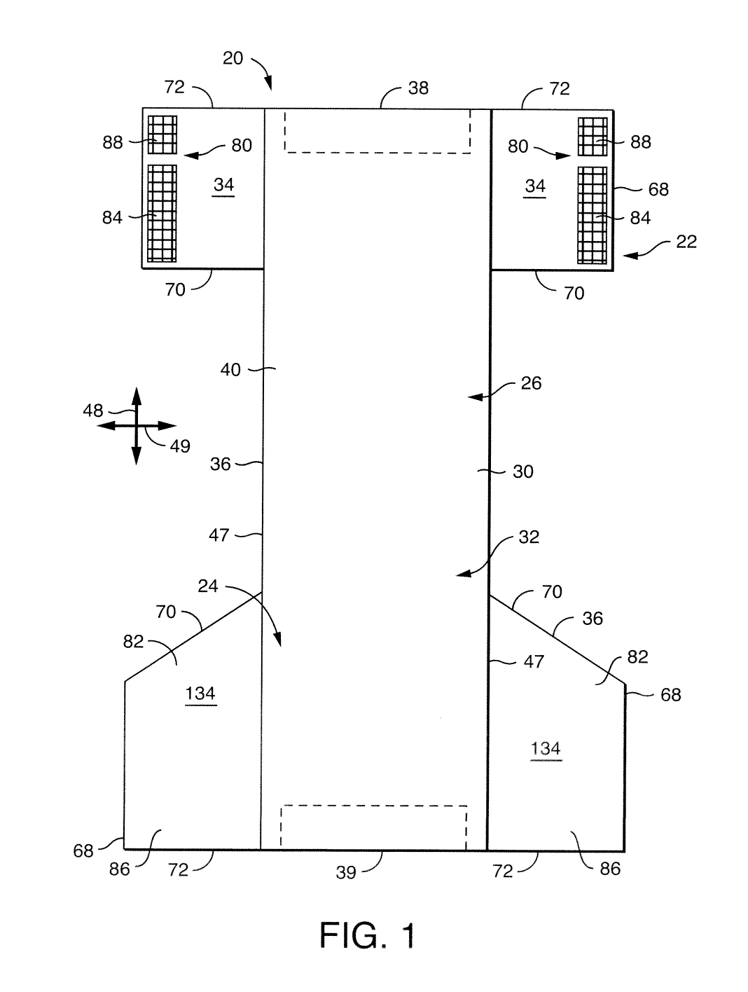

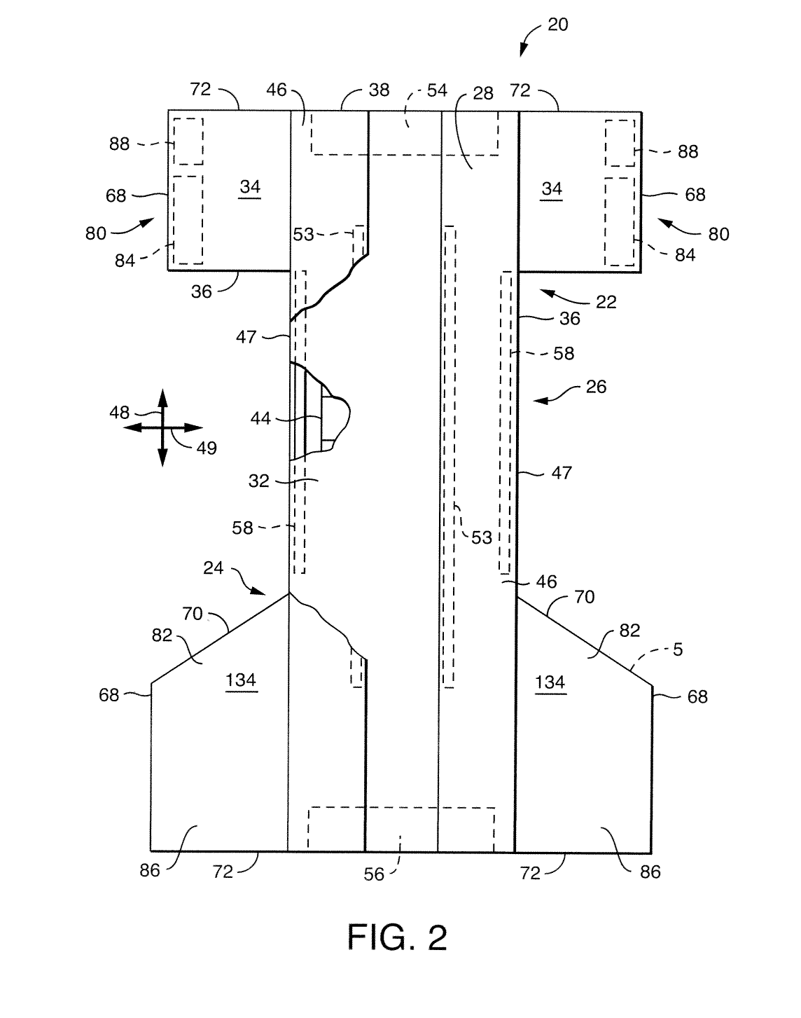

“Cross direction assembly” refers to a process in which disposable absorbent products are manufactured in an orientation in which the products are connected side-to-side, in the transverse direction shown by arrow 49 in FIGS. 1 and 2, a process utilizing a cross direction assembly that entails products traveling through a converting machine parallel to the direction of arrow 49, as opposed to “machine direction assembly” in which the products are connected end-to-end or waist-to-waist.

“Disposable” refers to articles that are designed to be discarded after a limited use rather than being laundered or otherwise restored for reuse.

“Disposed,” “disposed on,” and variations thereof are intended to mean that one element can be integral with another element, or that one element can be a separate structure bonded to or placed with or placed near another element.

“Elastic,” “elasticized” and “elasticity” mean that property of a material or composite by virtue of which it tends to recover its original size and shape after removal of a force causing a deformation.

“Elastomeric” refers to a material or composite that can be elongated by at least 25 percent of its relaxed length and that will recover, upon release of the applied force, at least 10 percent of its elongation. It is generally preferred that the elastomeric material or composite be capable of being elongated by at least 100 percent, more preferably by at least 300 percent, of its relaxed length and recover, upon release of an applied force, at least 50 percent of its elongation.

“Fabrics” is used to refer to all of the woven, knitted and nonwoven fibrous webs.

“Film” refers to a thermoplastic film made using a film extrusion and/or foaming process, such as a cast film or blown film extrusion process. The term includes apertured films, slit films, and other porous films that constitute liquid transfer films, as well as films that do not transfer liquid.

“Flexible” refers to materials that are compliant and that will readily conform to the general shape and contours of the wearer's body.

“Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by a Cahn SFA-222 Surface Force Analyzer System, or a substantially equivalent system. When measured with this system, fibers having contact angles less than 90 are designated “wettable” or hydrophilic, while fibers having contact angles greater than 90 are designated “nonwettable” or hydrophobic.

“Integral” or “integrally” is used to refer to various portions of a single unitary element rather than separate structures bonded to or placed with or placed near one another.

“Layer” when used in the singular can have the dual meaning of a single element or a plurality of elements.

“Liquid impermeable,” when used in describing a layer or multi-layer laminate, means that a liquid, such as urine, will not pass through the layer or laminate, under ordinary use conditions, in a direction generally perpendicular to the plane of the layer or laminate at the point of liquid contact. Liquid, or urine, can spread or be transported parallel to the plane of the liquid impermeable layer or laminate, but this is not considered to be within the meaning of “liquid impermeable” when used herein.

“Liquid permeable material” or “liquid water-permeable material” refers to a material present in one or more layers, such as a film, nonwoven fabric, or open-celled foam, which is porous, and which is water permeable due to the flow of water and other aqueous liquids through the pores. The pores in the film or foam, or spaces between fibers or filaments in a nonwoven web, are large enough and frequent enough to permit leakage and flow of liquid water through the material.

“Longitudinal” and “transverse” have their customary meaning, as indicated by the longitudinal and transverse axes depicted in FIGS. 1 and 2. The longitudinal axis lies in the plane of the article and is generally parallel to a vertical plane that bisects a standing wearer into left and right body halves when the article is worn. The transverse axis lies in the plane of the article generally perpendicular to the longitudinal axis. The article as illustrated is longer in the longitudinal direction than in the transverse direction.

“Machine direction” refers to the length of a fabric in the direction in which it is produced, as opposed to “cross direction” that refers to the width of a fabric in a direction generally perpendicular to the machine direction.

“Machine direction assembly” refers to a process in which disposable absorbent products are manufactured in an orientation in which the products are connected end-to-end or waist-to-waist, in the longitudinal direction shown by arrow 48 in FIGS. 1 and 2, a process utilizing a machine direction assembly entails products traveling through a converting machine parallel to the direction of arrow 48, as opposed to “cross direction assembly” in which the products are connected side-to-side.

“Meltblown fiber” means fibers formed by extruding a molten thermoplastic material through a plurality of fine, usually circular, die capillaries as molten threads or filaments into converging high velocity heated gas (e.g., air) streams that attenuate the filaments of molten thermoplastic material to reduce their diameter, which can be to microfiber diameter. Thereafter, the meltblown fibers are carried by the high velocity gas stream and are deposited on a collecting surface to form a web of randomly dispersed meltblown fibers. Such a process is disclosed for example, in U.S. Pat. No. 3,849,241 to Butin et al. Meltblown fibers are microfibers that can be continuous or discontinuous, are generally smaller than about 0.6 denier, and are generally self bonding when deposited onto a collecting surface. Meltblown fibers used in the present disclosure are preferably substantially continuous in length.

“Member” when used in the singular can have the dual meaning of a single element or a plurality of elements.

“Nonwoven” and “nonwoven web” refer to materials and webs of material that are formed without the aid of a textile weaving or knitting process.

“Operatively joined,” in reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joinder. The joining, attaching, connecting or the like can be either directly, such as joining either member directly to an element, or can be indirectly by means of another member disposed between the first member and the first element.

“Peel force” and “peel strain” refer to forces that tend to pull two adjoining bodies away from one another in opposite directions generally perpendicular to a plane in which the bodies are joined.

“Permanently bonded” refers to the joining, adhering, connecting, attaching, or the like, of two elements of an absorbent garment such that the elements tend to be and remain bonded during normal use conditions of the absorbent garment.

“Polymers” include, but are not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to isotactic, syndiotactic and atactic symmetries.

“Refastenable” refers to the property of two elements being capable of releasable attachment, separation, and subsequent releasable reattachment without substantial permanent deformation or rupture.

“Releasably attached,” “releasably engaged,” and variations thereof refer to two elements being connected or connectable such that the elements tend to remain connected absent a separation force applied to one or both of the elements, and the elements being capable of separation without substantial permanent deformation or rupture. The required separation force is typically beyond that encountered while wearing the absorbent garment. It should be noted that a releasably attached or releasably engaged seam is a refastenable seam that does not include a bonded seam that must be torn, cut, or otherwise disrupted.

“Shearing strain” refers to forces that tend to produce an opposite but parallel sliding motion between two bodies' planes.

“Spunbonded fiber” refers to small diameter fibers that are formed by extruding molten thermoplastic material as filaments from a plurality of fine capillaries of a spinnerette having a circular or other configuration, with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Petersen, and U.S. Pat. No. 3,542,615 to Dobo et al., each of which is incorporated herein in its entirety by reference. Spunbond fibers are quenched and generally not tacky when they are deposited onto a collecting surface. Spunbond fibers are generally continuous and often have average deniers larger than about 0.3, more particularly, between about 0.6 and 10.

“Stretchable” means that a material can be stretched, without breaking, to at least 150% of its initial (unstretched) length in at least one direction, suitably to at least 200% of its initial length, desirably to at least 250% of its initial length.

“Superabsorbent” or “superabsorbent material” refers to a water-swellable, water-insoluble organic or inorganic material capable, under the most favorable conditions, of absorbing at least about 15 times its weight and, more desirably, at least about 30 times its weight in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds such as cross-linked polymers.

“Surface” includes any layer, film, woven, nonwoven, laminate, composite, or the like, whether pervious or impervious to air, gas, and/or liquids.

“Thermoplastic” describes a material that softens when exposed to heat and that substantially returns to a nonsoftened condition when cooled to room temperature.

These terms can be defined with additional language in the remaining portions of the specification.

Referring now to the drawings and in particular to FIGS. 1 and 2, a personal wear absorbent article according to one aspect is illustrated in the form of a pants-type article for wear about a wearer's waist, and more particularly in the form of children's toilet training pants, indicated in its entirety by the reference numeral 20. The term absorbent generally refers to articles that can be placed against or in proximity to the body of the wearer to absorb and/or retain various liquid wastes discharged from the body. The absorbent article can be disposable, which refers to articles that are intended to be discarded after a limited period of use instead of being laundered or otherwise restored for reuse. It is understood that the concepts described herein are suitable for use with various other pants-type articles such as adult incontinence articles, as well as other articles intended for personal wear such as clothing, diapers, feminine hygiene products, medical garments, surgical pads and bandages, other personal care or health care garments, and the like without departing from the scope of the present disclosure.

By way of illustration only, various materials and methods for constructing the training pants 20 are disclosed in PCT Patent Application WO 00/37009 published Jun. 29, 2000 by A. Fletcher et al; U.S. Pat. No. 4,940,464 issued Jul. 10, 1990 to Van Gompel et al.; and U.S. Pat. No. 5,766,389 issued Jun. 16, 1998 to Brandon et al., which are incorporated herein by reference.

The present disclosure is directed to a pant-like absorbent garment having refastenable fasteners, such as hook and loop fasteners, on the side panels for ease of removal and donning of the absorbent garment. The side seam of the refastenable side seam training pants is designed with an engagement strength sufficient to keep the side seam/pant closed during wear. When a caregiver grasps a side panel to open the side seam, the caregiver must apply enough force to overcome the engagement strength of the side seam. The engagement side seam shear strength likely represents the maximum force required to open the side seam during use.

What this means for training pant design is that the strength of a refastenable side seam does not need to be uniform along its longitudinal length as long as the total strength is sufficient to prevent pop-opens during use. The side seam engagement strength can be lower in portions without negatively impacting the frequency of side seam pop-opens, as long as the side seam engagement strength is higher in other portions to compensate. A lower required side seam engagement strength in portions means that those portions of the side seam fasteners can use less fastening material such as less hook material, less aggressive hook material, lower bond strengths, fewer bond points, less adhesive, or some combination of these and other factors together referred to herein as lighter hook. Conversely, hook with increases in these factors is referred to herein as heavier hook.

The fasteners of the present application allow lighter hook to be used in locations prone to skin irritation due to pop-opens, seam misalignment, and the like, while heavier hook can be used in other locations to maintain overall seam strength. In a particular aspect of the present application, the portions of the side seam fasteners nearest the waist opening of the training pants 20, where closure/pop-open forces are potentially the greatest, can include heavier hook as an anchor to ensure the refastenable side seam remains fastened during use. Using a small portion of the side seam fasteners as an anchor allows for the majority of the side seam fasteners to include lighter hook to keep those portions of the side seam closed. Because the anchor fastener carries a higher load per unit area, the closure fastener carries a smaller load, and can therefore include a lighter, more skin friendly hook material. As a result, any misalignment of the majority of the side seam (the entire side seam except for that including the anchor fastener) that exposes the closure fastener to the wearer's skin will result in less skin irritation because of the more skin friendly hook made possible by this improvement.

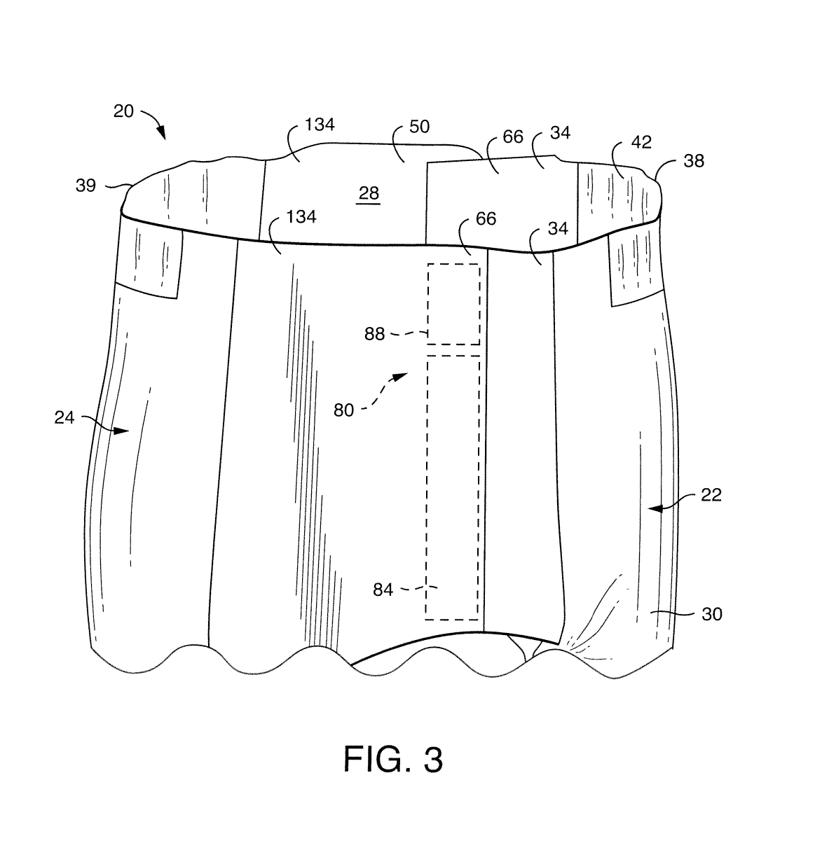

The pair of training pants 20 is illustrated in FIG. 3 in a fully pre-assembled (i.e., as assembled during initial manufacture) configuration (broadly referred to herein as a wear configuration of the pants, i.e., absorbent article). With additional reference to FIGS. 1 and 2, the training pants 20 includes a front waist region 22, a back waist region 24, a crotch region 26 extending longitudinally between and interconnecting the front and back waist regions 22, 24 along a longitudinal direction of the pants, an inner surface 28 configured for contiguous relationship with the wearer, and an outer surface 30 opposite the inner surface 28. The training pants 20 also has a pair of laterally opposite side edges 36 and a pair of longitudinally opposite waist edges, respectively designated front waist edge 38 and back waist edge 39. The front waist region 22 is contiguous with the front waist edge 38, and the back waist region 24 is contiguous with the back waist edge 39.

The illustrated pants 20 includes a central absorbent assembly, generally indicated at 32, which when laid flat as in FIGS. 1 and 2 can be rectangular or any other desired shape. A pair of laterally opposite front side panels 34 extends outward from the absorbent assembly 32 at the front waist region 22 (thereby forming transversely outer portions of the front waist region 22, and more broadly in part forming transversely opposite sides of the training pants). Laterally opposite back side panels 134 extend outward from the absorbent assembly 32 at the back waist region 24 (thereby forming transversely outer portions of the back waist region 24, and together with the front side panels 34 further defining the sides of the pants).

The central absorbent assembly 32 of the illustrated aspect includes an outer cover 40 and a bodyside liner 42 (FIG. 3) connected to the outer cover 40 in a superposed relation by suitable means such as adhesives, ultrasonic bonds, thermal bonds or other conventional techniques. An absorbent structure 44 (FIG. 2) is disposed between the outer cover 40 and the bodyside liner 42. A pair of containment flaps 46 (FIG. 2) is secured to the bodyside liner 42 for inhibiting the lateral flow of body exudates. The central absorbent assembly 32 has opposite ends that form portions of the front and back waist edges 38 and 39, and opposite side edges 47 that form portions of the side edges 36 of the training pants 20 (FIGS. 1 and 2).

The absorbent assembly 32 and side panels 34, 134 can include two or more separate elements, as shown in FIGS. 1 and 2, or they can be integrally formed. Integrally formed side panels 34, 134 and absorbent assembly 32 would include at least some common materials, such as the bodyside liner 42, flap composite, outer cover 40, other materials and/or combinations thereof, and could define a one-piece elastic, stretchable, or nonstretchable pants 20. For further reference, arrows 48 and 49 in FIGS. 1 and 2 depict the orientation of a longitudinal axis and a transverse or lateral axis, respectively, of the training pants 20.

With the training pants 20 in the fastened condition as illustrated in FIG. 3, the front and back side panels 34, 134 are attached to each other by a primary, or article fastening system 80 to define the pre-assembled three-dimensional wear configuration of the pants 20, having a waist opening 50 and a pair of leg openings. The front waist region 22 includes the portion of the training pants 20 that, when worn, is positioned at least in part on the front of the wearer while the back waist region 24 includes the portion of the training pants 20 that is positioned at least in part on the back of the wearer. The crotch region 26 of the training pants 20 includes the portion of the training pants 20 that is positioned between the legs of the wearer and covers the lower torso of the wearer.

The front and back side panels 34 and 134 include the portions of the training pants 20 (and more particularly of the front and back waist regions 22, 24) that, when worn, are positioned on the hips of the wearer. The attached side panels 34, 134 thus broadly define the transversely opposite sides of the pants 20 at an engagement seam 66 along which the fastening system 80 releasably attaches the front and back side panels 34, 134. The waist edges 38 and 39 of the training pants 20 are configured to encircle the waist of the wearer and together define the waist opening 50 (FIG. 3). Portions of the side edges 36 in the crotch region 26 generally define leg openings. The side panels 34 and 134 desirably have a panel length dimension measured parallel to the longitudinal axis 48 along the outer edge 68.

The central absorbent assembly 32 is configured to contain and/or absorb exudates discharged from the wearer. For example, the containment flaps 46 are configured to provide a barrier to the transverse flow of body exudates. A flap elastic member 53 (FIG. 2) can be operatively joined with each containment flap 46 in any suitable manner as is well known in the art. The elasticized containment flaps 46 define a partially unattached edge that assumes an upright configuration in at least the crotch region 26 of the training pants 20 to form a seal against the wearer's body. The containment flaps 46 can be located along the side edges 36 of the pants 20, and can extend longitudinally along the entire length of the absorbent assembly 32 or can only extend partially along the length of the absorbent assembly 32. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the training pants 20 also suitably includes a front waist elastic member 54 (FIG. 2), a rear waist elastic member 56, and leg elastic members 58, as are known to those skilled in the art. The waist elastic members 54 and 56 can be attached to the outer cover 40 and/or the bodyside liner 42 along the opposite waist edges 38 and 39, and can extend over part or all of the waist edges. The leg elastic members 58 can be attached to the outer cover 40 and/or the bodyside liner 42 along the opposite side edges 36 and positioned in the crotch region 26 of the training pants 20. The leg elastic members 58 can be longitudinally aligned along each side edge 47 of the absorbent assembly 32.

The outer cover 40 suitably includes a material that is substantially liquid impermeable. The outer cover 40 can be a single layer of liquid impermeable material, but more suitably includes a multi-layered laminate structure in which at least one of the layers is liquid impermeable. The inner layer of the outer cover 40 can be both liquid and vapor impermeable, or it can be liquid impermeable and vapor permeable.

It is also contemplated that the outer cover 40 can be stretchable, and more suitably elastic. In particular, the outer cover 40 is suitably stretchable and more suitably elastic in at least the transverse or circumferential direction of the pants 20. In other aspects the outer cover 40 can be stretchable, and more suitably elastic, in both the transverse and the longitudinal direction.

The liquid permeable bodyside liner 42 is illustrated as overlying the outer cover 40 and absorbent structure 44, and can, but need not, have the same dimensions as the outer cover 40. The bodyside liner 42 is suitably compliant, soft feeling, and non-irritating to the child's skin. Further, the bodyside liner 42 can be less hydrophilic than the absorbent structure 44 to present a relatively dry surface to the wearer and permit liquid to readily penetrate through its thickness.

Alternatively, the bodyside liner 42 can be more hydrophilic or can have essentially the same affinity for moisture as the absorbent structure 44 to present a relatively wet surface to the wearer to increase the sensation of being wet. This wet sensation can be useful as a training aid. The hydrophilic/hydrophobic properties can be varied across the length, width and/or depth of the bodyside liner 42 and absorbent structure 44 to achieve the desired wetness sensation or leakage performance.

The bodyside liner 42 can also be stretchable, and more suitably elastic. In particular, the bodyside liner 42 is suitably stretchable and more suitably elastic in at least the transverse 49, or circumferential direction of the pants 20. In other aspects, the bodyside liner 42 can be stretchable, and more suitably elastic, in both the transverse 49 and the longitudinal 48 directions.

As noted previously, the illustrated training pants 20 have front and back side panels 34 and 134 defining transversely opposite sides of the pants 20 in the wear configuration of the pants 20. The side panels 34, 134 can be permanently attached along seams 66 to the central absorbent assembly 32 in the respective front and back waist regions 22 and 24. More particularly, as seen best in FIG. 1, the front side panels 34 can be permanently attached to and extend transversely outward beyond the side edges 47 of the absorbent assembly 32 in the front waist region 22, and the back side panels 134 can be permanently attached to and extend transversely outward beyond the side edges 47 of the absorbent assembly 32 in the back waist region 24. The side panels 34 and 134 can be attached to the absorbent assembly 32 using attachment means known to those skilled in the art such as adhesive, thermal, pressure or ultrasonic bonding. Alternatively, the side panels 34 and 134 can be formed as an integral portion of a component of the absorbent assembly 32. For example, the side panels 34, 134 can include a generally wider portion of the outer cover 40, the bodyside liner 42, and/or another component of the absorbent assembly 32.

The front and back side panels 34, 134 each have an outer edge 68 spaced laterally from the seam 66, a leg end edge 70 disposed toward the longitudinal center of the training pants 20, and a waist end edge 72 disposed toward a longitudinal end of the training pants 20. The leg end edge 70 and waist end edge 72 extend from the side edges 47 of the absorbent assembly 32 to the outer edges 68. The leg end edges 70 of the side panels 34 and 134 form part of the side edges 36 of the training pants 20. The leg end edges 70 of the illustrated aspect are suitably curved and/or angled relative to the transverse axis 49 to provide a better fit around the wearers legs. However, it is understood that only one of the leg end edges 70 can be curved or angled, such as the leg end edge 70 of the back waist region 24, or neither of the leg end edges 70 can be curved or angled, without departing from the scope of this disclosure. The waist end edges 72 are suitably parallel to the transverse axis 49. The waist end edges 72 of the front side panels 34 form part of the front waist edge 38 of the training pants 20, and the waist end edges 72 of the back side panels 134 form part of the back waist edge 39 of the pants 20.

The side panels 34, 134 suitably, although not necessarily, include a stretchable material capable of stretching in a direction generally parallel to the transverse axis 49 of the training pants 20. More suitably the side panels 34, 134 include an elastic material. Suitable elastic materials, as well as one process of incorporating stretchable side panels into training pants, are described in the following U.S. Pat. Nos. 4,940,464 issued Jul. 10, 1990 to Van Gompel et al.; 5,224,405 issued Jul. 6, 1993 to Pohjola; 5,104,116 issued Apr. 14, 1992 to Pohjola; and 5,046,272 issued Sep. 10, 1991 to Vogt et al.; all of which are incorporated herein by reference. In particular aspects, the stretch material can include a stretch-thermal laminate (STL), a neck-bonded laminate (NBL), a reversibly necked laminate, or a stretch-bonded laminate (SBL) material. Methods of making such materials are well known to those skilled in the art and described in U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT application WO 01/88245 in the name of Welch et al.; all of which are incorporated herein by reference. Other suitable materials are described in U.S. patent application Ser. Nos. 12/649,508 to Welch et al. and 12/023,447 to Lake et al., all of which are incorporated herein by reference.

Alternatively, the side panel material can include other woven or nonwoven materials, such as those described above as being suitable for the outer cover 40 or bodyside liner 42; mechanically pre-strained composites; or stretchable but inelastic materials.

The absorbent structure 44 can be any structure that is generally compressible, conformable, non-irritating to the wearers skin and capable of absorbing and retaining liquid body exudates, and can be manufactured in a wide variety of sizes and shapes, and from a wide variety of absorbent materials commonly used in the art.

The article fastening system 80 includes laterally opposite closure components 84 adapted for refastenable engagement to corresponding closure landing zones 82. The article fastening system 80 also includes laterally opposite anchor components 88 adapted for refastenable engagement to corresponding anchor landing zones 86. In one aspect, a front or outer surface of each of the closure and anchor components 84, 88 and landing zones 82, 86 includes a plurality of engaging elements. The engaging elements of the closure components 84 are adapted to repeatedly engage and disengage corresponding engaging elements of the closure landing zones 82, and the engaging elements of the anchor components 88 are adapted to repeatedly engage and disengage corresponding engaging elements of the anchor landing zones 86, to releasably secure the pants 20 in its three-dimensional configuration. The article fastening system 80 provides an attachment of a strength sufficient to maintain the article in a wear configuration during use of the pants 20 by the wearer.

The closure and anchor components 84, 88 can include any refastenable fasteners suitable for absorbent articles, such as adhesive fasteners, cohesive fasteners, mechanical fasteners, or the like. In particular aspects, the closure and anchor components 84, 88 include mechanical fastening components for improved performance. Suitable mechanical fastening components can be provided by interlocking geometric shaped materials, such as hooks, loops, bulbs, mushrooms, arrowheads, balls on stems, male and female mating components, buckles, snaps, or the like.

In the illustrated aspect, the landing zones 82, 86 (i.e., one on each side of the training pants 20) include loop fasteners and the closure and anchor components 84, 88 include complementary hook fasteners. Alternatively, the landing zones 82, 86 can include hook fasteners and the closure and anchor components 84, 88 can include complementary loop fasteners. In another aspect, the closure landing zones 82 (i.e., one on each side of the training pants 20) include loop fasteners and the closure components 84 include complementary hook fasteners, and the anchor components 88 can include loop fasteners and the anchor landing zones 86 can include complementary hook fasteners. In still another aspect, the closure and anchor components 84, 88 and landing zones 82, 86 can include interlocking similar surface fasteners, or adhesive and cohesive fastening elements such as an adhesive fastener and an adhesive-receptive landing zone or material; or the like.

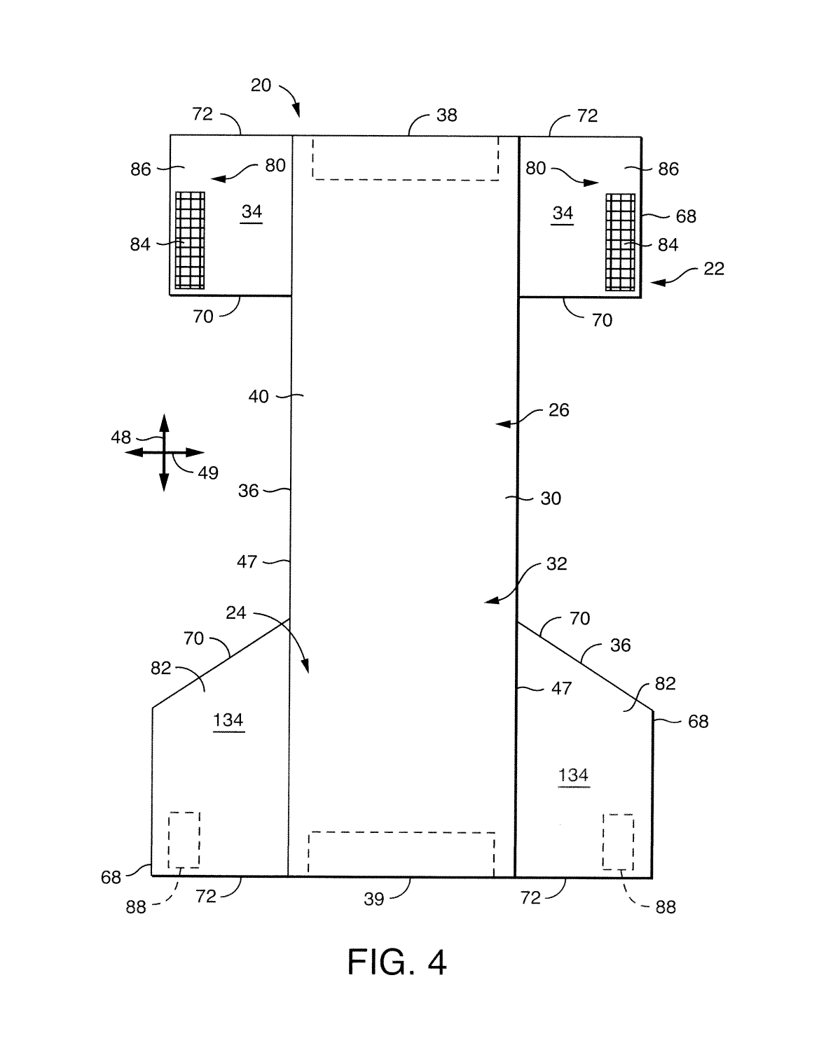

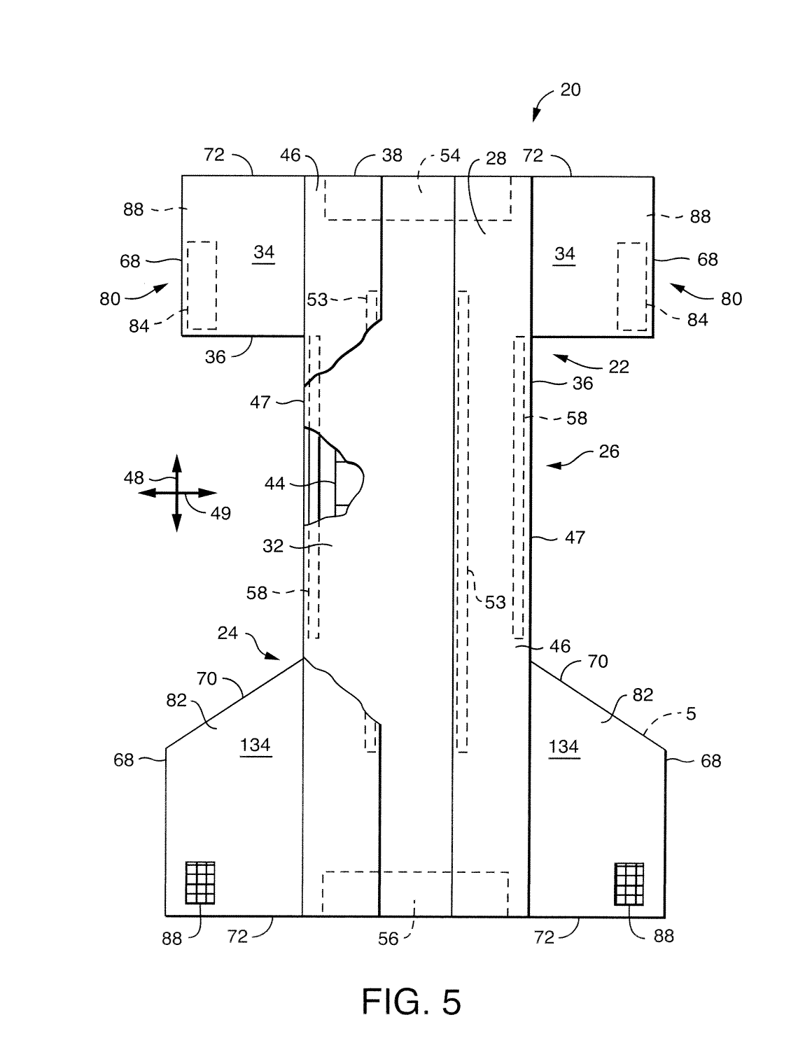

In another aspect of the present application illustrated in FIGS. 4 and 5, the anchor components 88 are hook material and are disposed on the inner surface of the back side panel 134, with the anchor landing zone 86 disposed on or integral with the outer surface of the front side panel 34. In this aspect, the closure component 84 is disposed on the outer surface of the front side panel 34, with the closure landing zone 82 disposed on the inner surface of the back side panel 134.

Loop type fasteners typically comprise a fabric or material having a base or backing structure and a plurality of loop members extending upwardly from at least one surface of the backing structure. The loop material can be formed of any suitable material, such as acrylic, nylon or polyester, and can be formed by methods such as warp knitting, stitch bonding or needle punching. Suitable loop materials are available from Guilford Mills, Inc., Greensboro, N.C., U.S.A. under the trade designation No. 36549. The loop material can be an integral portion of the outer surface or inner surface of the side panel rather than a separated material or structure.

Hook type fasteners typically comprise a fabric or material having a base or backing structure and a plurality of hook members extending upwardly from at least one surface of the backing structure. In contrast to the loop type fasteners which desirably comprise a flexible fabric, the hook material advantageously comprises a resilient material to minimize unintentional disengagement of the fastener components as a result of the hook material becoming deformed and catching on clothing or other items. The term “resilient” as used herein refers to an interlocking material having a predetermined shape and the property of the interlocking material to resume the predetermined shape after being engaged and disengaged from a mating, complementary interlocking material. Suitable hook material can be molded or extruded of nylon, polypropylene or another suitable material. Suitable single-sided hook materials for the fastening components 82 and 83 or the mating fastening components 84 and 85 are available from Velcro Industries B.V., Amsterdam, Netherlands or affiliates thereof, and are identified as Velcro HTH-829 with a uni-directional hook pattern and having a thickness of about 0.089 millimeters (3.5 mils) and HTH-851 with a uni-directional hook pattern and having a thickness of about 0.051 millimeters (2 mils).

Although the training pants 20 illustrated in FIG. 3 show the back side panels 134 overlapping the front side panels 34 upon connection thereto, which is convenient, the training pants 20 can also be configured so that the front side panels 34 overlap the back side panels 134 when connected. One skilled in the art will recognize that the shape, density, and polymer composition of the hooks and loops can be selected to obtain the desired level of engagement between the closure and anchor components 84, 88 and the landing zones 82, 86. A more aggressive or heavier hook material can include a material with a greater average hook height and/or a greater percentage of directionally-aligned hooks. When engaged, the closure and anchor components 84, 88 and landing zones 82, 86 of the illustrated aspect define the refastenable engagement seams 66 (FIG. 3).

As discussed above, in one particularly suitable aspect, as best seen in FIG. 2, the back side panels 134 are constructed so that the inner surfaces of the respective back side panels 134 define loop landing zones 82, 86 (i.e., the back side panels 134 and the landing zones 82, 86 are formed integrally). It is understood, however, that the loop landing zones 82, 86 can be formed separately from the back side panels 134 and attached thereto, such as by adhesive, thermal bonds, ultrasonic bonds, pressure bonds, or other suitable techniques without departing from the scope of this disclosure.

In one aspect of the present application, the closure and anchor components 84, 88 can be applied to the pants 20 as separate elements. In other words, a closure component 84 and an anchor component 88 are each applied to the front and/or back side panels 34, 134 on one transverse side of the pants 20. In this aspect, the complexity added by placing two components can be balanced by the lower complexity of separately manufacturing two elements including, for example, different hook materials.

In another aspect of the present application, the closure and anchor components 84, 88 can be manufactured together and applied to the pants 20 as one element. In this aspect, a web of hook material can be manufactured, for example, having machine-direction lanes of different hook materials. The web is then cut and applied to the pants 20 in a manner such that one set of lanes becomes the closure component 84 and the other set of lanes becomes the anchor component 88.

In both of these aspects, a cut-in-place type of operation well known in the business can place the closure and anchor components 84, 88 at the panel outer edge 68, either separately or as one discrete element. The closure and anchor components 84, 88 are desirably positioned along the outer edges 68 of the front or back side panels 34, 134. In certain aspects, for example, the closure and anchor components 84, 88 are located within about 2 centimeters, and more particularly within about 1 centimeter, of the outer edges 68, the waist end edges 72, and the leg end edges 70.

When connected, the closure and anchor components 84, 88 and the landing zones 82, 86 form a refastenable seam 66. In particular aspects, each of the closure and anchor components 84, 88 and the landing zones 82, 86 defines a length dimension aligned generally parallel with the longitudinal axis 48 of the training pant 20 and a width dimension aligned generally parallel with the transverse axis 49 of the training pant 20. For a child of about 9 to about 15 kilograms (20-30 pounds), for example, the length dimension of the closure and anchor components together is desirably from about 5 to about 13 centimeters, such as about 10 centimeters, and the width dimension is desirably from about 0.5 to about 3 centimeters, such as about 1 centimeter. The closure and anchor components 84, 88 desirably have a length-to-width ratio of about 2 or greater, such as about 2 to about 25, and particularly about 5 or greater, such as about 5 to about 8.

In particular aspects of the present application, each of the anchor components 88 defines a length dimension aligned generally parallel with the longitudinal axis 48 of the training pant 20 and a width dimension aligned generally parallel with the transverse axis 49 of the training pant 20. For example, the length dimension of the anchor components 88 is desirably from about 0.25 to about 4 centimeters, such as about 2 centimeters, and the width dimension is desirably from about 0.5 to about 3 centimeters, such as about 1 centimeter. The anchor components 88 desirably have a length-to-width ratio of about 2 or less, such as about 0.25 to about 1.9, and particularly about 1.5 or less, such as about 0.5 to about 1.

The closure components 84 can have a length dimension greater than about 50% of a length dimension of the outer edges 68 of the side panels 34, 134, specifically greater than about 60%, or greater than about 80% of the length dimension of the outer edges 68 of the side panels 34, 134. The anchor components 88 can have a length dimension less than about 40% of the length dimension of the outer edges 68 of the side panels 34, 134, specifically less than about 30%, less than 25%, less than 20%, or less than about 15% of the length dimension of the outer edges 68 of the side panels 34, 134.

In particular aspects of the present application, each of the closure components 84 defines an area, and each of the anchor components 88 defines an area. Further the ratio of the area of a closure component 84 to the area of an anchor component 88 can be greater than about 3:1, alternatively greater than about 5:1, or alternatively greater than about 6:1. This ratio is a result of the desire to have a secure but relatively discrete anchor fastener that is less likely to contact and irritate the skin.

With particular reference now to FIG. 3, an anchor component 88 is provided for use in fastening the side seam 66 of the training pants 20. As illustrated in FIG. 2, the article fastening system 80 includes an anchor component 88 attached to one of the front and back side panels 34, 134 (broadly, to the transversely opposite sides of the training pants 20). The anchor component 88 can be releasably attached to a side panel 34, 134 when the pants 20 is in a wear configuration.

The anchor component 88 fastens to the anchor landing zone 86 when the article 20 is in a wear configuration. In this aspect, the connection between the anchor component 88 and the anchor landing zone 86 has higher peel and shear strengths per unit area than the peel and shear strengths per unit area of the connection between the closure component 84 and the closure landing zone 82. Where the closure and anchor components 84, 88 include hook type fasteners disposed on the outer surface 30 and the landing zones 82, 86 include loop type fasteners disposed on the inner surface 28, the landing zones 82, 86 can be sized larger than the closure and anchor components 84, 88 to ensure coverage of the rigid hooks.

The use of outwardly-directed hooks for the closure components 84 can provide an advantage in that if the front side panel 34 and the back side panel 134 are misaligned, the skin of the wearer is more likely to be exposed to the inwardly-directed, relatively flexible loop of the closure landing zone 82 and less likely to be exposed to the outwardly-directed relatively more rigid hook. Because the anchor components 88 are primarily designed to secure the training pant 20 about the waist of the wearer, the size, shape, configuration and material design can be tailored for this specific use.

In a particularly preferred aspect of the present invention, the smaller anchor components 88, including heavier hook or otherwise-accomplished higher peel and shear strengths per unit area, are disposed on the outer surfaces 30 of the front side panels 34 adjacent the waist end edges 72 and the outer edges 68 of the front side panels 34. The larger closure components 84 are disposed adjacent the leg end edges 70 and the outer edges 68 of the front side panels 34. In a variation of this aspect, the smaller anchor components 88 are disposed on the inner surfaces 28 of the back side panels 134 adjacent the waist end edges 72 and the outer edges 68 of the back side panels 134.

In another aspect of the present disclosure, the difference in peel and shear strengths can be accomplished by adjusting the characteristics of the closure and anchor landing zones 82, 86. The closure landing zone 82, for example, can include a material having lower peel and shear strengths than the material of the anchor landing zone 86. The material of one landing zone can be differentiated by treatment of the side panel material, by selecting a different material for a portion of the side panel, or by adding material to the side panel in either or both of the landing zones 82, 86. For example, the material of the anchor landing zone 86 can be an added material coupled to the side panel, the side panel including side panel material, wherein the added material has higher peel and shear strengths than the side panel material.

In another aspect of the present disclosure, a given side panel can have an inner surface and an outer surface, where the inner surface has peel and shear strengths when used in conjunction with a given hook material that are different from the peel and shear strengths of the outer surface when used in conjunction with the same hook material. In an alternative aspect of the present disclosure, one of the front and back side panels 34, 134 has an inner surface 28, and the other of the front and back side panels 34, 134 has an outer surface 30. In this aspect, the inner surface 28 has peel and shear strengths when used in conjunction with a given hook material that are different from the peel and shear strengths of the outer surface 30 when used in conjunction with the same hook material. In these aspects, the anchor component 88 can fasten to one of the inner and outer surfaces 28, 30, with the closure component 84 fastening to another of the inner and outer surfaces 28, 30.

In an alternative aspect of the present disclosure, the differences in peel and shear strengths can be provided by using hook materials of different strengths or aggressiveness. For example, the anchor component 88 can have higher peel and shear strengths per unit area when engaged to a side panel material than the peel and shear strengths per unit area of the closure component 84 when engaged to the same side panel material.

In yet another aspect of the present disclosure, the differences in peel and shear strengths can be provided by using an increased amount of hook material for the anchor component 88.

In one aspect of the present application, the anchor component 88 of the illustrated article fastening system 80 includes a hook fastener. The anchor component 88 can include one, two, or more lanes or rows of hook material. The inner or outer surface of each front or back side panel 34, 134 suitably defines a corresponding fastening component, e.g., a loop fastener, to permit the anchor component 88 on each side of the pants 20 to be attached to the respective front or back side panel 34, 134 (i.e., broadly, to the pants 20) in the wear configuration of the pants 20. For example, the front side panel 34 in one particularly suitable aspect can be constructed of VFL material as described previously so that the inner and outer surfaces of the front side panel 34 itself defines a loop fastening component. Alternatively, a loop fastener component (not shown) can be formed separately from the front side panel 34 and attached to the panel outer surface without departing from the scope of this disclosure. The outer facing surface 30 of the outer cover 40 of the pants 20 is also suitably constructed to define a loop fastener, such as by forming the outer cover 40 of a material that defines a loop fastening component (e.g., VFL or other suitable material) or by forming a separate loop fastening component and attaching it to the outer surface 30 of the pants 20 outer cover 40, to permit attachment of the anchor component 88 to the outer cover 40 in the disposal configuration of the pants 20.

In an aspect of the present disclosure illustrated in FIG. 3, the article fastening system 80 can be arranged such that the closure component 84 and the anchor component 88 are generally colinear and are both generally equidistant from the longitudinal centerline when the article is in a wear configuration. Such an arrangement allows full use of the stretch inherent in the side panel material. In one example of this aspect, the closure component 84 extends only partially from the leg opening to the waist opening 50, leaving a longitudinal gap having no article fastening component. The anchor component 88 is disposed such that the anchor component 88 is positioned in the gap when the article 20 is in a wear configuration.

In use, the training pants 20 are constructed and pre-assembled in their wear configuration, with the article fastening system 80 releasably attaching the front and back waist regions 22, 24 (and more particularly the front and back side panels 34, 134 in the illustrated aspect). The anchor component 88 is releasably attached to the outer surface of the respective front side panel 34 in the wear configuration of the pants 20.

When the pants 20 are to be discarded after use, the pants 20 can be slipped off of the wearer in the manner of conventional underpants, or the front and back waist regions 22, 24 can be detached from each other (e.g., by separation of the article fastening components 84, 88 of the article fastening system 80) and the pants 20 removed from the wearer.

### Peel Strength Test

The level of securement of any particular area of the refastenable seam can be quantified using the following Peel Test method, which is designed to quantify, in grams, the peak and the average dynamic peel strength of the refastenable seam holding the front waist region 22 of the absorbent article 20 to the back waist region 24. The refastenable seam is used in this description as an example. The direction of removal or peel in this disclosure is that direction in which the fastener material would generally be removed from a substrate when the product is in use.

### Sample Preparation

The size of suitable samples of refastenable seam material having a single enhanced refastenable attachment zone can measure 7.6 cm. by 1.9 cm. (3 by 0.75 inch), with the enhanced refastenable attachment zone located 0.6 cm. (0.25 inch) inboard of one end edge. The size of the sample may need to be adjusted for different refastenable seam configurations.

### Equipment

1. Constant rate of extension tensile tester equipped with an appropriate load cell. A suitable tensile testing system is a Sintech Tensile Tester, commercially available from MTS, Research Triangle Park, N.C., under the trade designation Sintech Model 1/G Tensile Tester.
2. Software commercially obtained from MTS under the trade designation MTS TESTWORKS® for Windows Version 3.10.
3. Pneumatic-action grips commercially available from Instron Corporation, Canton, Mass., under the trade designation Instron Model 2712-004.
4. 2.5 cm. by 10.1 cm. (1 by 4 inch) grip faces, rubber coated, commercially available from Instron Corporation, Canton, Mass.
5. Test facility having a temperature of 23±1° C., and a relative humidity of 50±2 percent.

### Test Procedure

1. A sample to be tested is conditioned in the test facility for at least 4 hours prior to testing.
2. A 2041.2 grams (4.5 lb.) roller with a total diameter of 95 mm., the outer 6.7 mm. of which is rubber, is rolled over the sample from one end to the other and then back again (1 cycle).
3. The load cell is calibrated and the software loaded.
4. The grips are installed on the tensile tester with the jaws closed.
5. The test conditions for the tensile tester are set as follows:
Crosshead speed: 500 millimeters/minute
Full-scale load: 11.34 kilograms (25 lbs.)
Gage length: 25.4 millimeters (1 inch)
6. The weight of the clamp is tared out.
7. The sample is pulled apart on the end opposite from the enhanced refastenable attachment zone so that the fastening component and the mating fastening component disengage to form free ends each 25.4 millimeters long.
8. The free end of the fastening component on the back waist region of the article is inserted into the upper jaw.
9. The free end of the mating fastening component on the front waist region of the article is inserted into the lower jaw, such that the fastened inner surface of the back waist region and the fastened inner surface of the front waist region are facing the same direction and are parallel to one another. The lower jaw is closed.
10. The crosshead is started in motion, and the test is run until the fastening component and mating fastening component are no longer connected.
11. The average load needed to separate the fastener is recorded for the refastenable attachment zone by averaging load values at separation distances that avoid any enhanced refastenable attachment zone, for example from 1 cm. by 6.4 cm. (0.4 to 2.5 inch) for the sample specified in the Sample Preparation section above. The peak load needed to separate the fastening components is recorded for the refastenable attachment zone(s). Two or more tests may be needed to obtain values for the refastenable attachment zones.

### Shear Strength Test

This test method is designed to quantify, in grams, the peak dynamic shear strength of the releasable bonds assisting the primary fasteners in refastenably engaging the side panels to the front waist region of the absorbent article. The direction of force in this application is generally perpendicular to the longitudinal centerline of the product.

### Equipment

1. Tensile tester capable of obtaining a peak load and equipped with an appropriate load cell. A suitable tensile testing system is a Sintech Tensile Tester, commercially available from MTS Sintech, Research Triangle Park, N.C., under the trade designation Instron Model 4201 Tensile Tester with Sintech QAD (Quality Assurance Department) Software.

2. Software commercially obtained from MTS Sintech under the trade designation Sintech Testworks™.

3. Pneumatic-action grips commercially available from Instron Corporation, Canton, Mass., under the trade designation “Instron Model 2712-004.”

4. 1 by 4 inch grip faces, serrated, commercially available from Instron Corporation, Canton, Mass.

5. Test facility having a temperature of 23±1° C., and a relative humidity of 50±2 percent.

### Test Procedure

1. A sample to be tested is conditioned in the test facility for at least 4 hours prior to testing.

2. The load cell is calibrated and the software loaded.

3. The grips are installed on the tensile tester with the jaws closed.

4. The test conditions for the tensile tester are set as follows:

| | | |
|---|---|---|
| Crosshead speed | 500 | millimeters/minute |
| Full-scale load | 5 | kilograms |
| Threshold | 5 | percent |
| Fail criterion | 95 | percent |
| Gage length | 50 | millimeters |

5. The weight of the clamp is tared out.

6. The primary fastening element on the side panel of the article is inserted into the upper jaw such that the edge of the grip face is flush with the inner edge of the hook material.

7. The front waist region of the article is inserted into the lower jaw such that the inner surface of the side panel and the inner surface of the front waist region are facing the same direction and are parallel to one another. The lower jaw is closed.

8. The crosshead is started in motion.

9. The peak load of release is recorded.

When introducing elements of the present disclosure or the preferred aspect(s) thereof, the articles “a,” “an,” and “the” are intended to mean that there are one or more of the elements. The terms “comprising,” “including,” and “having” are intended to be inclusive and mean that there can be additional elements other than the listed elements.

The disclosure has been described with reference to various specific and illustrative aspects and techniques. However, it should be understood that many variations and modifications can be made while remaining within the spirit and scope of the disclosure. Many alternatives, modifications and variations will be apparent to those skilled in the art in light of the foregoing description. Accordingly, this disclosure is intended to embrace all such alternatives, modifications, and variations that fall within the spirit and scope of the appended claims.

We claim:

1. An absorbent article for personal wear about a wearer's waist, the article having a transverse axis and a longitudinal centerline, and comprising:
a liquid permeable inner surface for facing the wearer;
an outer surface for facing away from the wearer;
an absorbent body disposed therebetween;
a front waist region, a back waist region, and a crotch region extending longitudinally between and interconnecting the front and back waist regions;
a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening;
a closure component coupled to or integral with the front side panel and disposed adjacent the leg opening, an anchor component coupled to or integral with one of the front and back side panels and disposed adjacent the waist opening, a closure landing zone coupled to or integral with the back side panel, and an anchor landing zone coupled to or integral with the side panel opposite the anchor component, wherein the closure component is fastened to the closure landing zone to form a closure connection and the anchor component is fastened to the anchor landing zone to form an anchor connection when the article is in a wear configuration, wherein the refastenable seam includes the closure and anchor connections, and wherein the anchor connection has higher peel and shear strengths per unit area than the peel and shear strengths per unit area of the closure connection.

2. The article of claim 1, wherein the closure landing zone includes an added material coupled to the back side panel.

3. The article of claim 2, wherein the side panel includes side panel material, wherein the added material has higher peel and shear strengths than the side panel material when used with the same hook material.

4. The article of claim 1, wherein the anchor component is coupled to or integral with the back side panel.

5. The article of claim 1, wherein each of the front and back side panels has an inner surface and an outer surface, and wherein the anchor component is coupled to the inner surface of the back side panel, and wherein the closure component is coupled to the outer surface of the front side panel.

6. The article of claim 1, wherein the closure and anchor components are coupled to the front side panel.

7. The article of claim 1, wherein the closure and anchor components are coupled to the back side panel.

8. The article of claim 1, wherein the closure and anchor components are each separate and discrete elements.

9. The article of claim 1, wherein the closure and anchor components are portions of the same discrete element.

10. The article of claim 1, wherein the closure component extends only partially from the leg opening to the waist opening, leaving a longitudinal gap having no closure component, and wherein the anchor component is disposed in the gap when the article is in a wear configuration.

11. The article of claim 1, wherein the closure component includes hook material.

12. The article of claim 1, wherein the anchor component includes hook material.

13. The article of claim 1, wherein the closure component and the anchor component are generally colinear and are both generally equidistant from the longitudinal centerline when the article is in a wear configuration.

14. The article of claim 1, wherein the front side panel has an outer edge having a length dimension, and wherein the closure component has a length dimension greater than about 60% of the length dimension of the outer edge.

15. The article of claim 1, wherein the front and back side panels each have an outer edge having a length dimension, and wherein the anchor component has a length dimension less than about 40% of length dimension of one of the outer edges.

16. The article of claim 1, wherein the anchor component defines an area, and wherein the closure component defines an area with a ratio of 3:1 to the area of the anchor component.

17. An absorbent article for personal wear about a wearer's waist, the article having a transverse axis and a longitudinal centerline, and comprising:
a liquid permeable inner surface for facing the wearer;
an outer surface for facing away from the wearer;
an absorbent body disposed therebetween;
a front waist region, a back waist region, and a crotch region extending longitudinally between and interconnecting the front and back waist regions;
a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening, and wherein each of the front and back side panels has an inner surface and an outer surface;
an anchor component coupled to or integral with the inner surface of the back side panel, a closure component coupled to or integral with the outer surface of the front side panel, a closure landing zone coupled to or integral with the back side panel opposite the closure component, and an anchor landing zone coupled to or integral with the front side panel opposite the anchor component, wherein the closure component is fastened to the closure landing zone to form a closure connection and the anchor component is fastened to the anchor landing zone to form an anchor connection when the article is in a wear configuration, and wherein the anchor connection has higher peel and shear strengths per unit area than the peel and shear strengths per unit area of the closure connection.

18. The article of claim 17, wherein the refastenable seam includes the closure and anchor connections.

19. An absorbent article for personal wear about a wearer's waist, the article having a transverse axis and a longitudinal centerline, and comprising:
a liquid permeable inner surface for facing the wearer;
an outer surface for facing away from the wearer;
an absorbent body disposed therebetween;
a front waist region, a back waist region, and a crotch region extending longitudinally between and interconnecting the front and back waist regions;
a front side panel attached to the front waist region and a back side panel attached to the back waist region, the front and back side panels being releasably attachable at a refastenable seam to define a wear configuration of the absorbent article having a waist opening and a leg opening spaced from the waist opening, wherein the front and back side panels each extend from the waist opening to the leg opening;
a closure component coupled to or integral with the front side panel and disposed adjacent the leg opening, the closure component including closure hook material, an anchor component coupled to or integral with one of the front and back side panels and disposed adjacent the waist opening, the anchor component including anchor hook material, a closure landing zone coupled to or integral with the back side panel, and an anchor landing zone coupled to or integral with the side panel opposite the anchor component, wherein the closure hook material is lighter and more skin friendly than the anchor hook material.

20. The article of claim 19, wherein each of the front and back side panels has an inner surface and an outer surface, and wherein the anchor component is coupled to the inner surface of the back side panel, and wherein the closure component is coupled to the outer surface of the front side panel.
